(19) United States
(12) Patent Application Publication
(10) Pub. No.: US 2005/0064600 A1
(43) Pub. Date: Mar. 24, 2005

(54) Detection method and apparatus

(76) Inventors: Terry Clark, Wiltshire (GB); Andrew James Thomas, Wiltshire (GB); Neil Charles Shand, Wiltshire (GB); Brian John Webb, Wiltshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/497,584

(22) PCT Filed: Dec. 5, 2002
(86) PCT No.: PCT/GB02/05520

(30) Foreign Application Priority Data
Dec. 5, 2001 (GB) 0131126.5

(51) Int. Cl. G01N 33/00

(52) U.S. Cl. 436/106; 422/80

(57) ABSTRACT

The present invention provides a method for the detection of a material comprising ammonium nitrate and a sugar, the method comprising sensing for the presence of hydrogen isocyanate (HNCO). The present invention further provides an apparatus suitable for the examination of a material suspected of comprising ammonium nitrate and a sugar, the apparatus comprising heating means in thermal communication with a sample holder suitable for the containment of the material, the sample holder being in gaseous communication with sensing region, the apparatus being further provided with a means for inducing flow of gas from the sample holder to the sensing region and a means for sensing the presence of hydrogen isocyanate in the sensing region.

This invention relates to the field of chemical detection, and particularly to the field of detection of chemicals comprising ammonium nitrate.

Ammonium nitrate is available as a commonly-used fertiliser, but can be combined with various other substances, such as sugar, flour and fuel oil, to create explosives. These explosives are often referred to as home-made explosives, or HMEs. It is of prime importance to many security agencies to be able to detect HMEs and to differentiate between such explosive mixtures and ammonium nitrate.

Several methods have been developed to test for the presence of explosive mixtures of ammonium nitrate and sugar (hereinafter AN/S). One such method is to bum or decompose a sample suspected of comprising AN/S and sensing for the presence of nitrous oxide (N₂O). However, this test is not specific to AN/S mixtures, giving a false positive result for AN itself. Wet chemistry can be used to detect for the presence of ammonium, nitrate and sugar moieties, but this method is generally time-consuming and relatively insensitive. Such wet chemistry methods, some of which are capable of discriminating between AN/S and AN, can also give false positive readings for other commonly available ammonium-containing chemicals.

The method of the present invention addresses some of these problems. According to the present invention, a method for the detection of a material comprising ammonium nitrate and a sugar, the method comprising sensing for the presence of hydrogen isocyanate (HNCO). This provides an alternative method for the detection of AN/S-based explosive. The term “sugar” is taken to mean any sugar that is capable of reacting in the presence of ammonium nitrate to form HNCO. Examples of such sugars are glucose and sucrose.

The method preferably comprises heating the material to elevated temperature (preferably about 280° C.) and sensing for the presence of hydrogen isocyanate. The material is preferably heated in the presence of a chemically amphoteric material, such as a ceramic material.

The method further preferably comprises sensing for the presence of nitrous oxide (N₂O). Nitrous oxide is a signature of the presence of ammonium nitrate. Sensing for the presence of both HNCO and nitrous oxide reduces that likelihood of a false positive result that may arise by sensing for the presence of HNCO alone. Such false positives may arise from the combustion of certain plastics materials. It is preferred that the sensing for the presence of hydrogen isocyanate and optionally nitrous oxide is performed using one or more of infra-red spectroscopy, gas chromatography and mass spectrometry. Infra-red spectroscopy is the most preferred method.

The invention further provides a method for the detection of an explosive, the explosive not necessarily comprising ammonium nitrate and sugar, wherein the method comprises sensing for the presence of hydrogen isocyanate.

According to another aspect of the present invention, an apparatus suitable for the examination of a material suspected of comprising ammonium nitrate and a sugar, the apparatus comprising heating means in thermal communication with a sample holder suitable for the containment of the material, the sample holder being in gaseous communication with a sensing region, the apparatus being further provided with a means for inducing flow of gas from the sample holder to the sensing region and a means for sensing the presence of hydrogen isocyanate in the sensing region.

This permits the fast and reliable detection of a material comprising AN/S mixtures such as HMEs.

It is preferred that the sensing region forms part of a sensing chamber. This is a chamber where gases can accumulate for testing.

It is preferred that both the sample holder and sensing chamber are substantially inert to hydrogen isocyanate. It is preferred that the sensing chamber is made from polytetrafluoroethylene (PTFE). At least part of the surface of the sensing chamber may be coated with gold. It has been found that for more efficient production of HNCO, the sample holder comprises a chemically amphoteric material, such as a ceramic material. Macor® (Corning, USA) is an example of such a ceramic material.

At least one, and preferably both, of the sample holder and sensing chamber may be readily removable from the apparatus. This facilitates cleaning and replacement of units. The sensing chamber is preferably of a modular form such that it can be readily deconstructed for cleaning.

It is preferred that a particle filter is placed in the gaseous path between the sample holder and the sensing region. This prevents particulates from entering the sensing region.

It is preferred that the gaseous communication between the sample holder and sensing region is provided by a substantially chemically-inert conduit. Such a conduit is resistant to the corrosive effects of HNCO and does not readily adsorb HNCO or other reaction products. It is preferred that the conduit is made from polytetrafluoroethylene (PTFE).

The apparatus may be provided with an inlet for allowing gas to be drawn into the sample holder prior to being drawn into the sensing region. Air is drawn into the sample holder which then carries the contents of the sample holder, including the HNCO, into the sensing region. A filter for the removal of carbon dioxide is preferably placed in the gaseous path between the inlet and the sample holder. This removes substantially all of the carbon dioxide from the carrier gas such that any carbon dioxide detected in the sample holder can be identified as originating from the sample being analysed. Alternatively, the carbon dioxide filter can be a chamber of such a volume that it acts as a reservoir, buffering the sample gas against external changes in the carbon dioxide concentration.

The apparatus preferably further comprises means for sensing the flow of gas through at least part of the apparatus. The apparatus may further comprise actuating means, responsive to the means for sensing the flow of gas, for controlling the means for sensing the presence of HNCO in the sensing region. In such an embodiment, the actuating means controls the timing of the operation of the HNCO sensor relative to the flow of sample into the sensing region.

It is preferred that the means for sensing the presence of hydrogen isocyanate comprises an infra-red light source and a detector. This provides a simple, effective and rapid detection apparatus. A suitable optic filter may be placed in the light path between the light source and the detector.

Such an apparatus may be used to examine explosives, not necessarily comprising ammonium nitrate and sugar, that liberate HNCO under suitable conditions.

The present invention will now be described, by way of example only, with reference to the following figures of which:

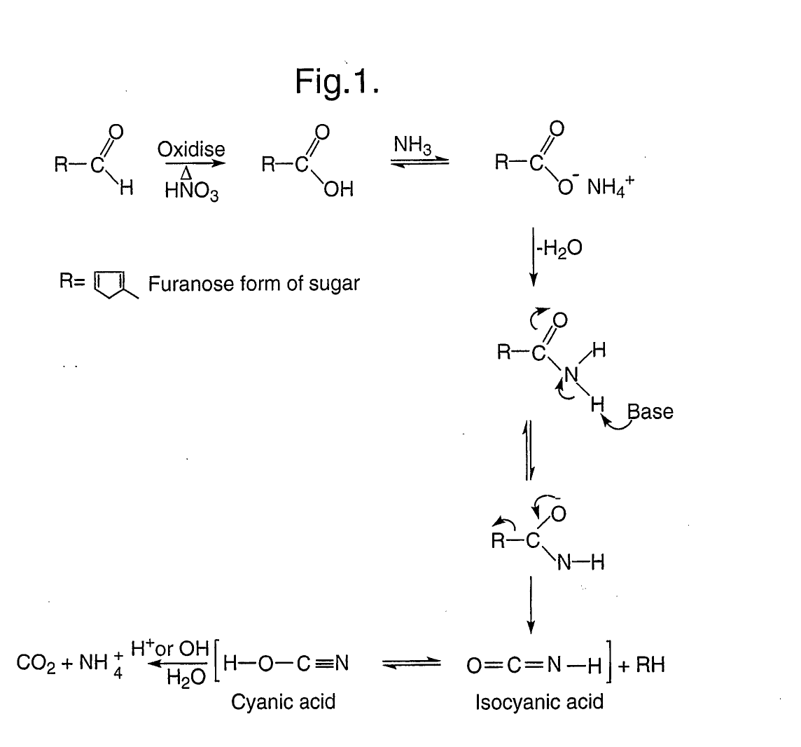

FIG. 1 is a proposed reaction scheme showing how HNCO may be generated by a sugar and ammonium nitrate;

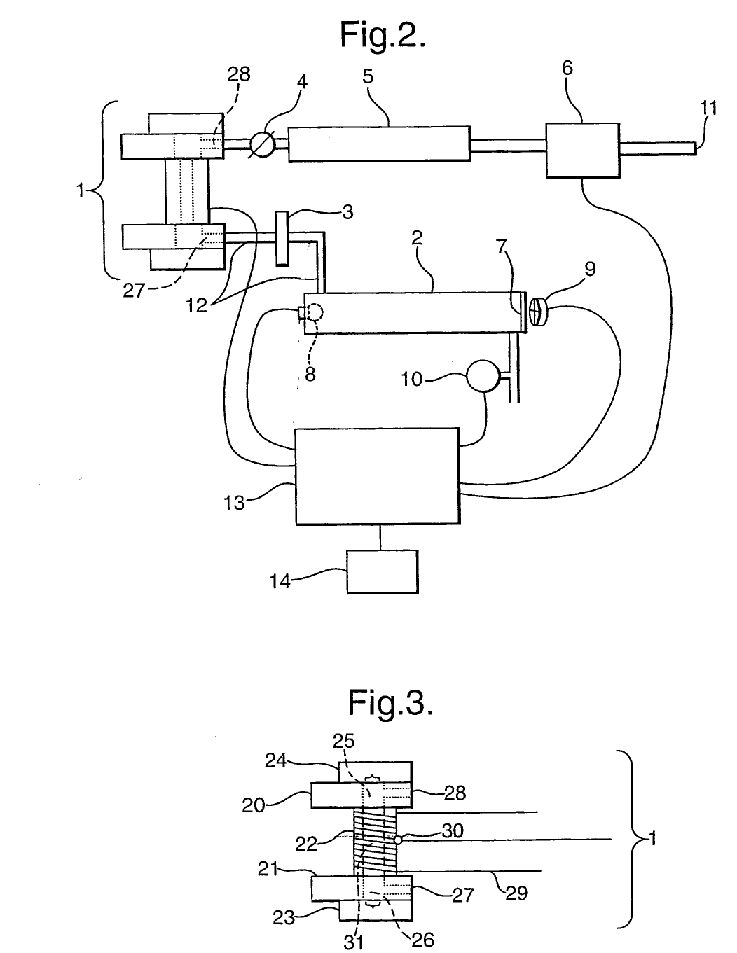

FIG. 2 is a schematic representation of an apparatus in accordance with the present invention;

FIG. 3 is a schematic representation of an oven assembly which forms part of the apparatus of FIG. 2;

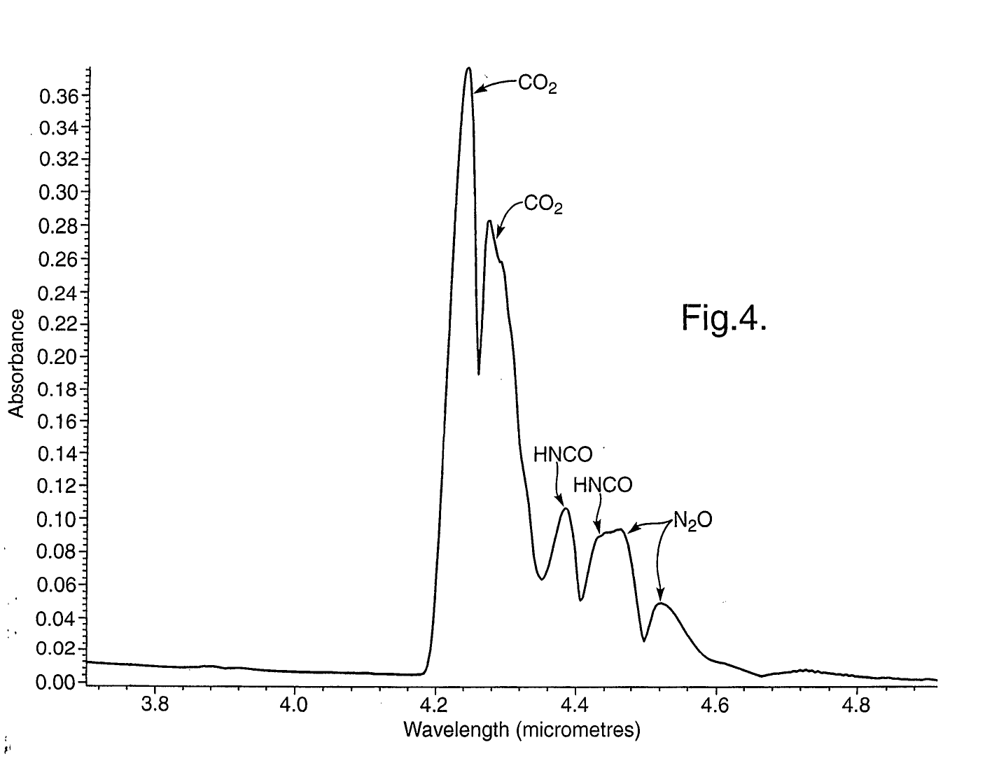

FIG. 4 is an infra-red absorbance spectrum generated by heating a sample of sugar and ammonium nitrate in an apparatus in accordance with the present invention;

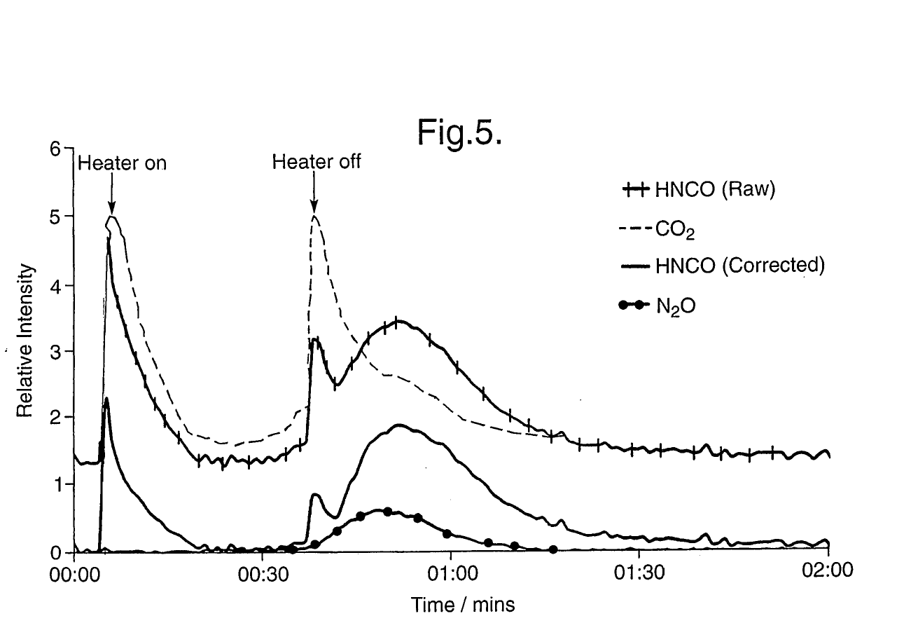

FIG. 5 is a graphical representation of the evolution over time of the infra-red absorption signals associated with HNCO, carbon dioxide and nitrous oxide generated by heating a sugar/ammonium nitrate sample in an apparatus in accordance with the present invention;

### METHOD OF THE PRESENT INVENTION

The applicants have discovered that, under certain well defined conditions, mixtures of ammonium nitrate and sugar react to produce hydrogen isocyanate (HNCO).

The proposed underlying chemistry of the reaction is given in the reaction scheme of FIG. 1.

Thus by sensing for the presence of hydrogen isocyanate, then one can determine whether the material under investigation; comprises ammonium nitrate and sugar (or a material that decomposes under the reaction conditions of the reaction scheme to liberate sugar). Note that, irrespective of the accuracy of the reaction scheme, the essential feature of the reaction scheme is that mixtures of sugar and ammonium nitrate generate HNCO.

The investigator may also wish to sense for the presence of nitrous oxide that would indicate the presence of ammonium nitrate. Testing for the presence of ammonium nitrate reduces the likelihood of false positive results arising from sensing for the presence of HNCO alone; it is possible that HNCO may be liberated by materials other then AN/S mixtures, such as certain plastics. Sensing for the presence of nitrous oxide indicates whether ammonium nitrate is present, something not likely to be present in the materials that alone may generate HNCO, such as plastics. Furthermore, the method according to the present invention may be enhanced by sensing for the presence of carbon dioxide, an indicator that organic matter (i.e. not ammonium nitrate) is contained within the sample. The amount of carbon dioxide would be indicative of whether the organic content of the sample could be considered as being significant. One may also develop the method of the present invention by sensing for the presence of unsaturated hydrocarbons, indicative of the presence of fuel oil, another possible component of HMEs.

The methods used to sense for the presence of HNCO and other compounds mentioned above will be well-known to those skilled in the art. Infra-red spectroscopy will be particularly apt, given that it is simple, fast and inexpensive. Relatively small spectrometers are available which may be used for this task.

### APPARATUS OF THE PRESENT INVENTION

FIG. 2 is a schematic representation of an apparatus in accordance with the present invention suitable for the examination of a material suspected of comprising ammonium nitrate and a sugar, the apparatus comprising an oven assembly 1, gas cell 2, particulate filter 3, flow control valve 4, carbon dioxide filter 5, gas pump 6, quartz window 7, infra-red light source 8, infra-red detector 9, pressure sensor 10, air inlet 11, sample gas tubing 12, control electronics 13 and display 14.

The gas pump 6 draws air through the air inlet 11 into the gas inlet port 28 of the oven assembly 1 via the carbon dioxide filter 5 and flow control valve 4. The air acts as a carrier gas. The carbon dioxide filter 5 removes substantially all of the atmospheric carbon dioxide; this is beneficial if the apparatus is used to detect the presence of carbon dioxide in the decomposition products of a sample. The inclusion of the flow control valve 4 is preferred since it allows the control of the flow of air through the oven assembly 1. Air is drawn through the gas inlet port 28 into the body of the oven assembly 1. In use, the sample contained within the oven assembly 1 is heated to 280° C., then allowed to cool. It has been found that this is a successful heating regime for the production of HNCO from AN/S mixtures. The reaction products are carried in the stream of air out of the body of the oven assembly 1 via a gas outlet port 27. The gas pump 6 acts as a means for inducing flow of gas to the sensing region in gas cell 2. However, those skilled in the art will realise that the air inlet 11 is not an essential integer of the present invention, merely preferable. The carrier gas and reaction products are passed along the sample gas tubing 12 into the gas cell 2 via the particulate filter 3. The particulate filter 3 removes particulate from the gas stream. Such particulate may comprise ammonium nitrate particles which may form from the gaseous reaction products nitrous oxide and ammonia. Infra-red light source 8 and infra-red detector 9 are arranged such that the infra-red adsorption characteristics of the contents of the gas cell 2 may be measured. The infra-red light source 8 in this case is a broad band source; one could alternatively use several narrow band or monochromatic sources. The infra-red detector 9 is isolated from the contents of the gas cell 2 by an inert, infra-red transparent quart window 7. The isolation of the detector 9 is preferable since HNCO is reactive and corrosive. The detector 9 is a four channel detector with the channels being tuned to the characteristic absorptions of a reference band and three important products of the AN/S mixtures which are generated when AN/S is decomposed in accordance with the method of the present reaction. The key components and the related adsorption bands are: carbon dioxide −4.24 μm, HNCO −4.4 μm, Nitrous oxide −4.5-4.55 μm, reference −3.95 μm. Each channel is provided with an appropriate optical band pass filter. Whilst it is desirable to identify many reaction products, those skilled in the art would realise that it is not essential to detect anything other than HNCO in the case of the present invention.

The pressure sensor 10 is in gaseous communication with the gas cell 2 and is further in communication with the control electronics 13. The preferred inclusion of the pressure sensor 10 ensures that a flow of air may be maintained through the apparatus. The control electronics 13 can be of any sort known to those skilled in the art and are further in communication with the gas pump 6, infra-red light source 8, infra-red detector 9 and oven assembly 1. The control electronics 13 controls and co-ordinates reaction product generation and data collection processes in any manner known to those skilled in the art. The control electronics 13 further cause the results of the analysis to be displayed on display 14. Those skilled in the art will realise that the apparatus may be operated manually without the use of the control electronics 13. The display 14 typically comprises a liquid crystal display. When a sample is being analysed a 17-bit display in the form of a bar is used for each of nitrous oxide, carbon dioxide, hydrogen isocyanate and hydrocarbon to indicate the presence of those species, the length of the bar being indicative of the amount of species present. Algorithms in the electronics 13 are used to analyse the data obtained from the sample to determine which one of four outcomes is displayed after the sample has been analysed; a—no AN/S present ; b—AN present; c—AN/S possibly present, try larger sample; d—AN/S present.

Some of the products of the decomposition of AN/S mixtures in accordance with the method of the present invention, in particular HNCO, are highly corrosive and reactive. It is highly preferred that any surface coming into contact with such chemicals is substantially inert to those chemicals. This increases the lifetime of the components bearing such surfaces and also provides an apparatus that gives a more accurate reading of the amount of HNCO released from a sample. It is preferred that sample gas tubing 12, parts of the oven assembly 1 and the particulate filter 3 comprise polytetrafluoroethylene (PTFE). PTFE is relatively inert to HNCO and has little effect on the compostion of the reaction product gas. Silicon rubber tubing should not be used for sample gas tubing 12 since it has a considerable effect on the amount of HNCO in the product gas stream. Furthermore, it is strongly preferred that the components which come into contact with HNCO should not be metal, although relatively inert metals such as gold are acceptable.

The gas cell 2 is a NDIR (non-dispersive infra-red) gas spectrometer cell. Such a cell is advantageous since it is provided with a substantially inert gold coating (not shown) on the surface of the cell that is in contact with the reaction products. The gas cell 2 is easily removed from the apparatus and is of a modular form such that it may be readily taken apart and reconstructed by the user to facilitate cleaning. Ease of removal and modularity are strongly preferred since ammonia and nitrous oxide may react, forming ammonium nitrate solid on the walls of the gas cell 2, thus causing a decrease in the sensitivity of the apparatus. The use of the particulate filter 3 helps in preventing ammonium nitrate particulates from reaching the gas cell 2.

Those skilled in the art will realise that the gas pump may be replaced by any means of drawing air through the apparatus. Such a means may use either positive pressure (e.g. pump, fan) or negative pressure (vacuum pump). A vacuum pump would ideally be placed downstream of the oven assembly 1 and gas cell 2. The gas pump 6 may be operated for a given period after a sample has been examined in order to flush material from the gaseous path of the apparatus. This minimises the risk of cross-contamination between samples.

Those skilled in the art will realise that the presence of gas cell 2 is strongly preferred when using infra-red radiation to identify reaction products. Such a chamber is not necessary, however. Furthermore, if using other detection techniques (such as mass spectrometry), then the use of a sensing chamber may not be preferred; the output of the oven assembly 1 may be passed directly into a spectrometer or alternative means of analysis.

The infra-red detector 9 preferably comprises a capability of detecting C—H bond stretch in addition to, or in place of, the capability of generating the reference signal. The apparatus may be arranged such that if the reference signal falls below a predetermined level, then the display 14 indicates that this has occurred and that the gas cell 2 requires cleaning.

The apparatus may further comprise a contamination sensor (not shown) that senses the accumulation of contaminants within the apparatus. Such a contamination sensor is preferably in communication with the control electronics 13.

The pressure sensor 10 may be replaced by a flow sensor. Those skilled in the art will realise that whilst preferable, the pressure sensor 10 is not essential to operation of the apparatus.

FIG. 3 is a schematic representation of an oven assembly 1 used in the apparatus of FIG. 2. The oven assembly 1 comprises supports 20, 21 for a ceramic heater 22 having a lumen 31 formed therein, sample cell closures 23, 24, entry port 25 formed in support 20, exit port 26 formed in support 21, gas inlet port 28, gas outlet port 27, NiCr heating wire 29 and thermocouple 30. The ceramic heater 22 is a machined Macor® (Corning, USA) component having a rectangular central lumen 31 formed therethrough. The lumen 31 extends the length of the heater 22. The NiCr heating wire 29 is wound around substantially the whole length of the heater 22 and is held in place by alumina cement (not shown) designed to operate at high temperatures. The thermocouple 30 is held in place by cement and is used as part of the temperature control mechanism for the oven assembly 1. A support 20, 21 is provided at each end of the heater 22, the supports 20, 21 being used to locate the oven assembly 1 within the apparatus of FIG. 1. The supports 20, 21 are made of PTFE and are each provided with a cylindrical bore which acts as an entry port 25 and exit port 26 respectively for samples. The entry port 25 and exit port 26 allow passage of a sample into, and out of, the lumen 31 respectively. The entry port 25 and exit port 26 are typically cylindrical bores but may be any suitable cavity.

The support 20 is also provided with a gas inlet port 28 which typically takes the form of a cylindrical bore. The gas inlet port 28 forms a gaseous connection between the lumen 31 of the heater 22 and the air inlet 11 of the apparatus of FIG. 2 via the entry port 25. Support 21 is provided with a gas outlet port 27 which also typically takes the form of a cylindrical bore. The gas outlet port 27 forms a gaseous connection between the lumen 31 of the heater 22 and the gas cell 2 via the exit port 26 and sample gas tubing 12.

Sample cell closures 23, 24 are placed over supports 21, 20 respectively after insertion of a sample into the lumen 31 and before the initiation of the heating process. Closures 23, 24 prevent the escape of air which enters the oven assembly 1 via gas inlet port 28 and also prevent escape of reaction products generated by the heating of the sample. The apparatus may be provided with an interlock such that a suitable error message is displayed on the display 14 if the oven assembly 1 is not properly connected to the rest of the apparatus.

Those skilled in the art will realise that other heating arrangements are possible.

FIG. 4 shows infra-red spectra generated by heating an AN/S sample to 280° C. and allowing it to cool in an apparatus in accordance with the present invention. The detector is used to sense the presence of HNCO, carbon dioxide and nitrous oxide. The data of FIG. 4 show that HNCO, carbon dioxide and nitrous oxide are all present. This is consistent with the presence of ammonium nitrate and sugar in the tested sample. The peak at approximately 4.4 μm is used to assess whether, and optionally how much, HNCO is present. The HNCO peak at approximately 4.44 μm merges with a nitrous oxide absorbance and thus is preferably not used to indicate the presence of HNCO.

FIG. 5 shows the evolution of infra-red absorbance signals corresponding to the presence of HNCO, carbon dioxide and nitrous oxide over time when a AN/S sample is heated in an apparatus in accordance with the present invention. By the time the heater is turned off, the temperature in the heater is about 280° C. Note that the HNCO data may be corrected for the overlap of the HNCO peak with one of the carbon dioxide peaks (see FIG. 4). With reference to FIG. 5, the peaks in absorbance that are observed when the heater is turned on and off are merely experimental artefacts associated with the particular apparatus used to acquire the data. Thus, it has been shown that the method and apparatus of the present invention may be used to detect the presence of AN/S mixtures. A 1-2 mg AN/S sample produces satisfactory positive results in the apparatus of the present invention. Smaller samples may also produce acceptable results, but are less reliable. However, those skilled in the art will realise that there are many ways in which the sensitivity of the apparatus may be improved, for example, by reducing the gaseous volume of the apparatus (by reducing the volume of the lumen 31, gas cell 2 and sample gas tubing 12) and reducing the reactivity with HNCO of the components that come into contact with HNCO.

The apparatus of the present invention may be used to detect other forms of home made explosive such as those comprising ammonium nitrate and fuel oil (referred to as “ANFO”). Heating a sample of ANFO explosive in the apparatus of the present invention will generate nitrous oxide, carbon dioxide and at least one hydrocarbon. The hydrocarbon may be conveniently detected using IR spectroscopy.

1. A method of detecting a material comprising a mixture of ammonium nitrate and a sugar, comprising the step of determining the presence of hydrogen isocyanate:

2. A method according to claim 1, in which the determination step monitors one or more IR absorption bands characteristic to hydrogen isocyanate.

3. A method according to claim 1, comprising the preliminary step of heating the mixture towards 280° C.

4. A method according to claim 3, in which the heating step is performed in the presence of a chemically amphoteric material.

5. A method according to claim 4, in which the chemically amphoteric material comprises a ceramic.

6. A method according to claim 1, also comprising the step of determining the presence of nitrous oxide.

7. Apparatus for detecting a mixture of ammonium nitrate and a sugar, comprising heating means for heating a sample thereof in a sample holder and gas flow means conducting gas from the sample holder to a sensing means, wherein the sensing means comprise means monitoring one or more IR absorption bands characteristic to hydrogen isocyanate.

8. Apparatus according to claim 7, in which the sample holder comprises a chemically amphoteric material.

9. Apparatus according to claim 8, in which the chemically amphoteric material comprises a ceramic.

10. Apparatus according to claim 7, in which the gas flow means include filter means removing, or correcting for, carbon dioxide.

11. Apparatus according to claim 7, in which the gas sensing means comprise a gas sensing cell comprising, at least in part, a material that is substantially inert to hydrogen isocyanate.

12. Apparatus according to claim 7, in which the gas flow means comprises, at least in part, a material substantially inert to hydrogen isocyanate.

13. A method of detecting a material comprising a mixture of ammonium nitrate and a hydrocarbon, comprising the step of monitoring one or more IR absorption bands characteristic of nitrous oxide and one or more IR absorption bands characteristic of hydrocarbons.

14. A method according to claim 13, in which the hydrocarbon is fuel oil.
